(19) United States
(12) Patent Application Publication
(10) Pub. No.: US 2026/0315789 A1
(43) Pub. Date: Oct. 15, 2026

(54) Intracranial Lead Fixation Device

(71) Applicant: Skulpin Medical, Inc., San Clemente, CA (US)

(72) Inventors: Peter G. Davis, San Clemente, CA (US); Farrokh R. Farrokhi, San Clemente, CA (US); Ravut Chhit, San Clemente, CA (US); K. David Crockett, Aliso Viejo, CA (US)

(73) Assignee: Skulpin Medical, Inc., San Clemente, CA (US)

(21) Appl. No.: 19/467,110

(22) Filed: Feb. 2, 2026

Related U.S. Application Data
(60) Provisional application No. 63/752,830, filed on Feb. 2, 2025.

(51) Int. Cl. A61N 1/05

(52) U.S. Cl. CPC A61N 1/0539

(57) ABSTRACT

Devices and methods described below provide for secure anchoring of an intracranial lead in the brain of a patient. The device includes an anchoring portion and a locking portion which can be installed in a burr hole, and an intracranial lead can be passed through the device, and then the locking portion can be rotated within the anchoring portion to bind the lead within the device. The device may be configured to allow installation in the burr hole so that the superficial surface is flush with the superficial surface of the skull, to avoid undesired bulges under the skin.

This application claims priority to U.S. Provisional Application 63/752,830, filed Feb. 2, 2025, the entirety of which is hereby incorporated by reference.

### FIELD OF THE INVENTIONS

The inventions described below relate to the field of intracranial lead fixation.

### BACKGROUND OF THE INVENTIONS

Intracranial leads are wires implanted in the brain, through the skull, for diagnosis or treatment of irregular brain activity. The lead can be used for brain stimulation (DBS). These leads must be located in specific areas of the brain which are known to affect various conditions resulting from abnormal brain activity. These conditions currently include Parkinson's disease, epilepsy, essential tremor and other conditions. Proper location of the leads is important because very specific areas of the brain are involved in each condition, and misplacement of the lead can result in misdiagnosis and unwanted effects of the stimulation. For some treatments, the leads are installed for extended periods or permanently, and it is important to prevent unwanted dislocation of the leads.

### SUMMARY

The devices and methods described below provide for secure anchoring of an intracranial lead in the brain of a patient. The device comprises an anchoring portion and a locking portion which can be installed in a burr hole, and an intracranial lead can be passed through the device, and then the locking portion can be rotated within the anchoring portion to bind the lead within the device. The anchoring portion is configured for secure direct insertion and attachment to the skull, and the locking portion is configured for secure insertion into the anchoring portion and attachment to the anchoring portion. The locking portion includes internal cam lobes that, upon rotation within the anchoring portion, impact prongs within the anchoring portion to deflect the prongs and clamp a lead passing through the device between the prongs. The device may be configured to allow installation in the burr hole so that the superficial surface is flush with the superficial surface of the skull, to avoid undesired bulges or depressions under the skin which create a risk of scalp irritation, erosion and infection. The device can also be installed without the need for fasteners (screws, nails or staples), thus avoiding the additional injury attendant to use of fasteners.

While developed in the context of leads for deep brain stimulation, which may installed for long periods, the Fixation Device can also be used to securely place other elongate device in the brain, including cannulas, drainage tubes, and other probes. Thus, the descriptions below apply, mutatis mutandis, to placement and fixation of any similar device.

### BRIEF DESCRIPTION OF THE DRAWINGS

FIG. 1 illustrates the head of a patient with a lead installed in the brain through a burr hole in the skull.

FIGS. 2, 3 and 4 illustrate the anchoring portion of the intracranial lead fixation device of FIG. 1.

FIGS. 5, 6, 7 and 8 illustrate the locking portion of the intracranial lead fixation device of FIG. 1.

FIGS. 9, 10, 11 and 12 illustrate the assembled intracranial lead fixation device of FIG. 1.

FIGS. 13 and 14 illustrate insertion and removal tools for use with the notched version of the anchoring portion and locking portion of the device.

### DETAILED DESCRIPTION OF THE INVENTIONS

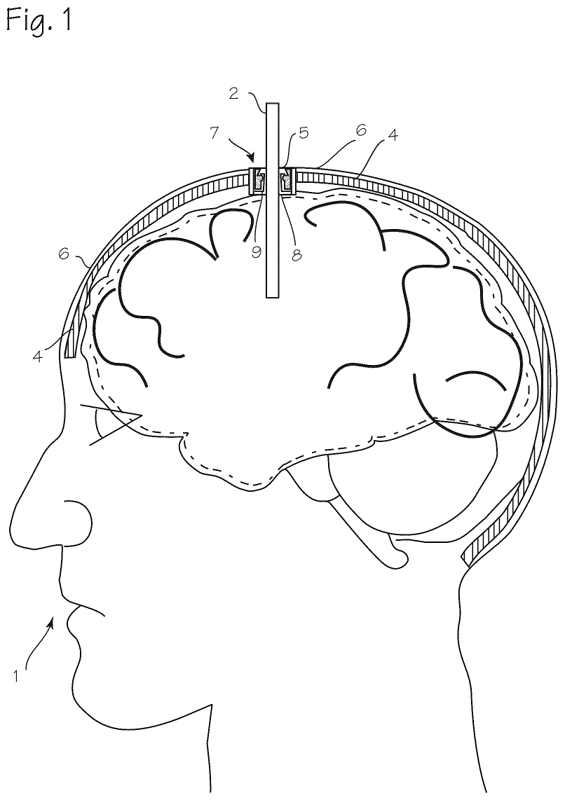

FIG. 1 illustrates a patient 1 with a lead 2 installed in the brain 3, fixed to the skull 4 through a intracranial lead fixation device 5. A surgeon has created an opening through the patient's scalp 6 and skull 4 with a burr hole 7. This figure depicts installation of a intracranial lead fixation device 5 into the skull to provide access of the lead 2 into the brain. The intracranial lead fixation device 5 comprises the anchoring portion 8 and the locking portion 9. The anchoring portion 8 is configured, as detailed below, to be fixed within the burr hole, securely to prevent or inhibit rotation of the anchoring portion within the burr hole, and translation within the burr hole. The locking portion 9 is configured for insertion into the anchoring portion, and rotation within the locking portion, to cause radially inward deflection of prongs such that the prongs will clamp the lead 2 passing through the anchoring portion.

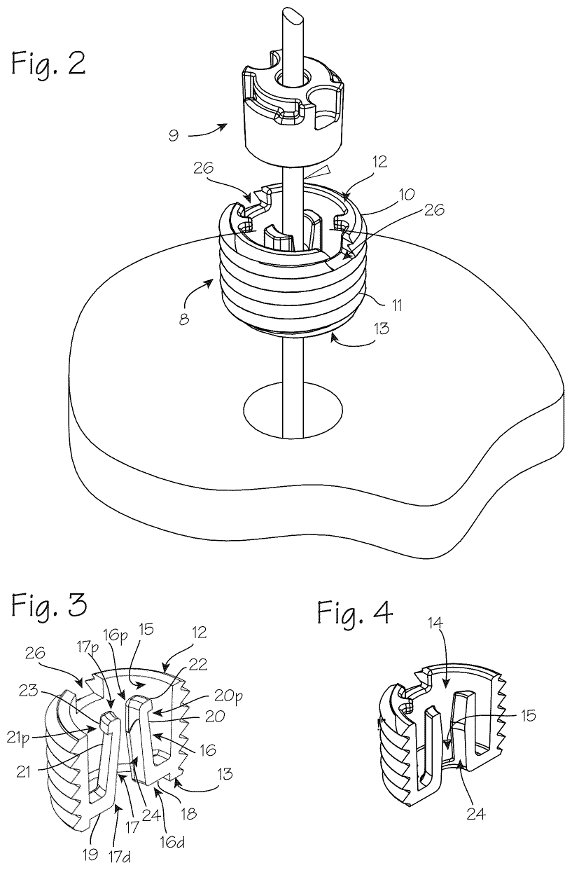

FIGS. 2 through 7 illustrate details of the device. FIGS. 2, 3 and 4 illustrate the anchoring portion 9. The anchoring portion 8 comprises a cylinder 10, with external threads 11, a proximal rim 12 (the rim disposed superiorly, near the outer surface of the skull when installed) and a distal rim 13 (the rim disposed inferiorly, near the inner surface of the skull when installed), and a lumen 14 extending through the anchoring portion from the proximal rim and the distal rim and establishing a luminal wall 15. The height (measured from the proximal rim to the distal rim) is preferably the same as the thickness of the skull or slightly less than the thickness of the skull of the particular patient. Preferably, the height of the anchoring portion is not more than the thickness of the skull surrounding the burr hole, so that the anchoring portion can be installed flush with the exterior surface of the skull.

The external threads provide a means for fixing the anchoring portion to the skull, with sufficient securement to prevent or impede translation or rotation of the anchoring portion. The threads are preferably self-tapping, so the is no need to create internal threads in the bore hole Securement need not be absolutely fixed or permanent: it is sufficient that securement prevent rotation and translation under the forces applied in use as described below. Other means for fixing the anchoring portion may be used, including snap-fit fittings on the outer and inner (proximal and distal) rims of the anchoring portion, press fit between the outer surface and the bore hole walls (with or without threads or flanges), or an adhesive or fasteners applied to the outer surface or a flange about the proximal rim. With these alternative means for fixing the anchoring portion, the transverse cross section of the anchoring portion need not be circular, so long as the burr hole is created to match the shape of the outer surface of the anchoring portion.

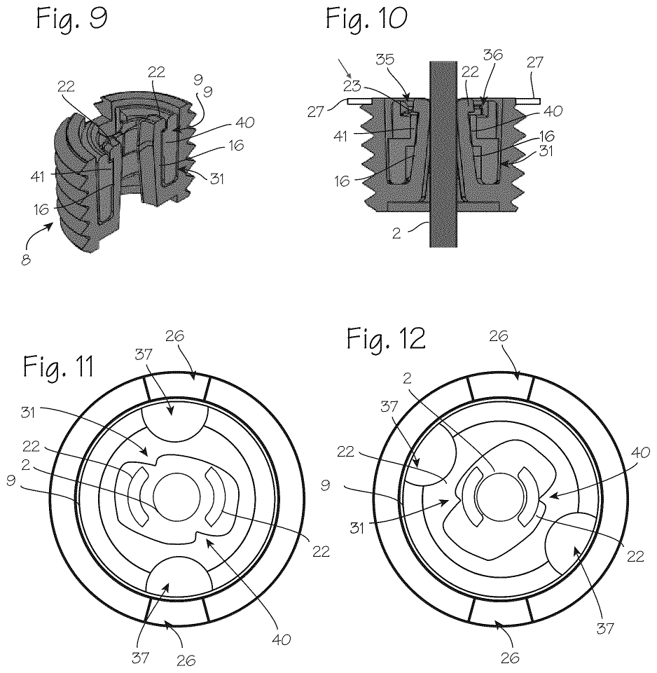

Within the lumen of the anchoring portion, a pair of prongs 16, 17 are fixed to the luminal wall 15 and extend longitudinally within the lumen 14 of the anchoring portion, from the distal end 10d of the anchoring portion toward the proximal end 10p of the anchoring portion. These may extend directly from the luminal wall, or they may extend from radially inwardly projecting supports 18, 19 near the distal end of the anchoring portion and the distal end of the prongs (FIG. 3). The prongs can also be bent to apply great pressure during clamping. The supports, or a direct join between the prongs and the luminal wall provide a mechanical stop for the insertion of the locking portion (see FIGS. 9 and 10). Preferably, the prong proximal ends 16p, 17p terminate proximally at the same level as the proximal rim 12 of the anchoring portion. The prongs illustrated comprise a locking means, characterized by the beams 20, 21 (the long, longitudinally extending portion) and the catch 22, 23 (the short, radially outwardly extending protrusions at the unsupported proximal ends 20p, 21p of each beam). As illustrated in FIGS. 9 and 10, the catches are configured to fit into receiving pockets in the inner wall of the locking portion, to longitudinally lock the locking portion to the anchoring portion. The catches may be omitted (as in FIG. 4) if the locking portion is configured to be secured longitudinally with the anchoring portion through other means, such as a friction fit or a bayonet fitting arrangement (using pins extending from the inner wall of the anchoring portion into a slot in the locking portion, or vice-versa). The beams of the prongs are deformable, and configured to bend, at the proximal end (corresponding the proximal end of the anchoring portion), so that they may impinge upon a lead passing through the device when the locking portion is inserted into the anchoring portion. The beams may be flexible and resilient, so that they open to release the lead when inward force of the locking mechanism, as described below, is removed. Preferably, the inner surface 24 of each beam is shaped to conform to the outer contour of the lead. For a round lead, the inner surface of the beam may be concave, along its length, to accommodate the round lead. This is shown in FIGS. 3, 4 and 11. FIG. 4 illustrates a variation of the anchoring portion structure, in which the catches are omitted and the beams extend from an inwardly extending flange 25 at the distal end of the anchoring portion The flange partially encloses the lumen 14 to limit fluid passing out of the brain to the external skull.

The device is illustrated in a preferred embodiment, using two prongs to clamp the lead, and two lobes or minor diameter regions to bend the prongs. Other means may be used to trap the lead, such as a single prong configured to press the lead, and a single large lobe sized to bend the single prong against an opposing portion of the locking portion.

To ease in insertion of screw-in embodiments, the anchoring portion includes a first pair (or more) of wrench notches 26, configured to receive the prongs of an insertion tool such as the prong wrench described below, with which the anchoring portion may be rotated and screwed into the burr hole. Embodiments which depend on press-fit of the anchoring portion into the burr hole may include an outwardly extending flange 27 on the outside of the proximal rim to prevent over-insertion, in which case the notches may be retained so that an insertion tool can be used to prevent rotation during locking rotation of the locking portion, or they may be omitted, and the threads may be retained or omitted. The flange 27 may be coupled with a releasable snap-fitting, such as an annular snap ring or releasable catches disposed at the distal end of the anchoring portion.

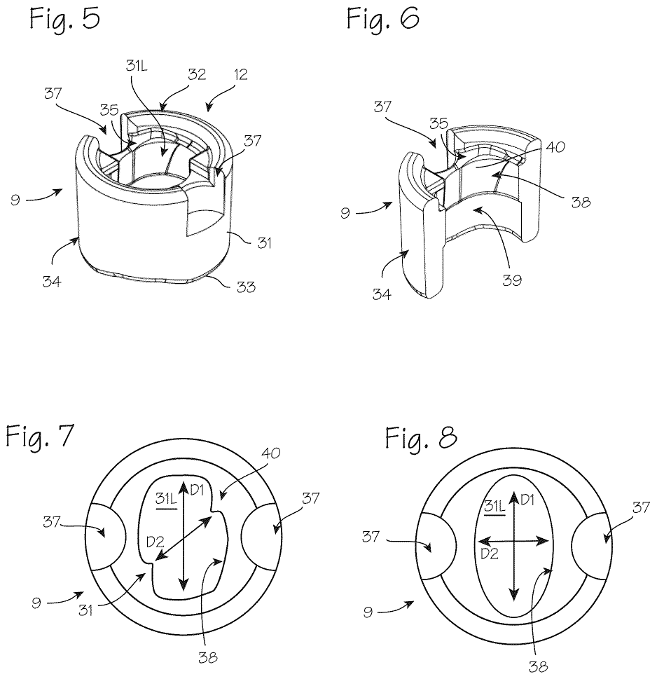

FIGS. 5, 6, 7 and 8 illustrate the locking portion 9 of the device, which includes a second cylinder 31 with a locking portion lumen 31L extending from the proximal end 31p and proximal rim 32 of the cylinder to the distal end 31p and distal rim 33 of the cylinder. The cylinder has a circular outer surface 34 and an outer diameter matching the inner diameter of the cylinder of the anchoring portion. The outer surface may include grooves or keyways, so long as the outer contour is rotatable within the anchoring portion. The locking portion has a wall thickness which allows it to be inserted into the anchoring portion such that the wall is disposed between the beams of the locking mechanism and the luminal wall of the anchoring portion. The inner wall of the locking portion includes pockets 35, 36 configured to receive the catches of the locking mechanism of the anchoring portion. (The catches and pockets may be omitted if other means for longitudinally locking the locking portion to the anchoring portion, such as a threaded fitting or bayonet fitting are used).

To provide for rotation of the locking portion, the locking portion includes a first pair (or more) of wrench notches 37, configured to receive the prongs of an insertion tool, with which the locking portions may be rotated within the anchoring portion. Embodiments which depend on press-fit of the locking portion, without rotation of the locking portion to engage the catches of the locking mechanism, may omit the notches.

FIG. 7 illustrates means by which the beams may be forced radially inwardly to impact a lead disposed within the lumen of the device. A proximal portion 38 of the luminal wall 39 of the locking mechanism includes lobes 40, 41, which establish a major transverse diameter D1 and a minor transverse diameter D2 smaller than the major diameter for the lumen. These establish a first circumferential region of large luminal diameter D1 and a second circumferential region of smaller luminal diameter D2. Upon initial insertion, the lobes must be positioned circumferentially between the beams, to allow insertion over the beams (with the space between the lobes providing for passage of the beams). After insertion, rotation of the locking portion will lead to impingement of the lobes with the proximal end of the beams, forcing the proximal ends of the beams radially inwardly and into contact with the lead. Thus, FIG. 7 illustrates a cam, distinguished from conventional cams in that the lobes are disposed within a hollow cylinder and act to convert rotary motion of the cylinder to inwardly radial movement of the beams. As an alternative to the lobes, the transverse cross section of the lumen 31L may be elliptical, as shown in FIG. 8, again with a major diameter D1 and a minor diameter D2 smaller than the major diameter, such that rotation of the locking portion can bring the minor diameter region of the wall of the lumen into impingement with the beams. Upon initial insertion, the minor diameter region must be positioned between the beams, to allow insertion over the beams (with the space between the lobes providing for passage of the beams). Thus, whether lobed or elliptical, the internal diameter is non-circular, with a region of minor diameter which can be brought into impingement with the beams by rotation of the locking mechanism within the anchoring mechanism after the locking portion is inserted with the major diameter region aligned with the beams so that the locking portion can fit into the anchoring portion.

The interaction of the locking portion, the anchoring means and the locking mechanism to provide a means for clamping the lead within the device are illustrated in FIGS. 9, 10 and 11. These figures show the locking portions inserted into the lumen of the anchoring portion and rotated slightly, with the catches 22,23 of the anchoring portion disposed within the pockets 35, 36 of the locking mechanism. (Note that the beams may flex inwardly to accommodate insertion of the locking mechanism, and thereafter the locking portion may be rotated until the pockets capture the catches). This arrangement prevents longitudinal, proximal movement of the locking portion out from the anchoring portion. The matching height of the locking mechanism and location of the beam join to the anchoring portion luminal wall also provides a stop which prevents over-insertion of the locking portion. The matching height of the locking mechanism and location of the beam join also results in a flush configuration, in which the proximal surface of both the anchoring portion and the locking portions may be installed flush with the skull with little protrusion showing through the scalp. The distance between the pockets and the distal edge of the locking mechanism matches the distance from the join to the catches, ensuring that catches are disposed longitudinally in line with the pocket upon full insertion of the locking portion into the anchoring portion.

FIGS. 9, 10, 11 and 12 illustrate the assembled device, with the locking portion installed in the anchoring portion. As shown in FIGS. 9 and 10, the cylinder 31 of the locking portion in disposed within the lumen 14 of the anchoring portion, with the cylinder 31 of the locking portion between the anchoring portion luminal wall 15 and the prongs 16. In FIG. 9 the locking portion is positioned with the lobes or minor diameter regions positioned between the prongs, and the prongs are widely spaced.

In FIGS. 10 and 12, the locking portion has been rotated so that the catches 22, 23 are disposed with the pockets 35, 36, and, concomitantly, the lobes 40, 41 have been rotated to impinge on the beams and the beams have been forced into contact with the lead. FIGS. 11 and 12 show a top view (proximal to the proximal end of the assembled device), in which the rotation of the lobes 40, 41 into contact with the beams is illustrated. In FIG. 11, the locking portion is disposed within the anchoring portion, with the major diameter accommodating the prongs and the prongs spaced apart, not in contact with the prongs. In FIG. 12, the locking portion has been rotated, to impinge on the proximal portion of the prongs to drive the prongs into contact with the lead.

Thus, the intracranial lead fixation device includes (1) the anchoring portion 8 having with the outer contour configured for placement in the burr hole, a distal end 10d and a proximal end 10p, and a circular lumen 14 extending from the distal end 10d to the proximal end 10p and a luminal wall 15 about the lumen 14, and a pair of prongs 16, 17 each having a prong distal end 16d, 17d and a prong proximal end 16p, 17p, wherein each prong distal end 16d, 17d is secured to the luminal wall proximate the distal end 10d of the anchoring portion 8 and each of the prongs 16, 17 comprise a beam 20, 21 which extends proximally within the lumen 14 of the anchoring portion 8 and terminating at the proximal end 10p of the anchoring portion 8, (2) the locking portion 9 with a circular outer contour configured for placement in the lumen of the anchoring portion, a distal end 31d and a proximal end 31p, and a locking portion lumen 31L extending from the distal end 31d to the proximal end 31p and a luminal wall 39 about the lumen 31L. The lumen 31L has a first circumferential region having a major transverse diameter D1 and a second circumferential region having a minor transverse diameter D2 smaller than the major transverse diameter D1. The locking portion is disposed within the lumen of the anchoring portion, and the prongs 16, 17 of the anchoring portion 8 are disposed within the lumen 31L of the locking portion 9. With this assembly, the locking portion may be rotated within the anchoring portion to move the second circumferential region into contact with each beam 20, 21 and force each beam to bend radially inwardly to trap a lead passing through the intracranial lead fixation device, and the locking portion may be counter-rotated to move the first circumferential region into apposition with the beams thereby allowing the beams to open and allow unimpeded passage of the lead through the intracranial lead fixation device.

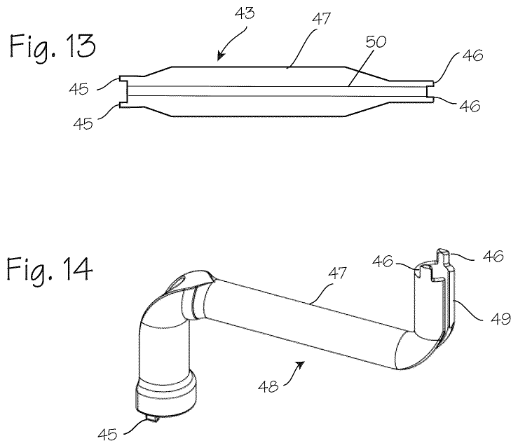

Handy insertion tools for use with the notched version of the anchoring portion and locking portion are shown in FIGS. 13 and 14. Each comprises a “prong wrench” with prongs spaced to match the notches of the two components. The prong wrenches 43, 44 resemble a drum wrench or golf spike wrench, with prongs 45, 46 extending from a handle portion 47, such that the prongs may be inserted into corresponding notches in the component to be rotated, parallel to the axis of rotation of the component, and the handle may be rotated or twisted about the axis of rotation to rotate the component. In the case of the anchoring portion, the end of the prong wrench with the prongs 45 spaced to match the notches of the anchoring portion is used to drive the anchoring portion with screw threads into the burr hole. In the case of the locking portion, the end of the prong wrench with prongs 46 spaced to match the notches of the locking portion is used, after insertion of the locking portion into the anchoring portion, to rotate the locking portion within the anchoring portion in order to bring the lobes of the internal cam structure into impingement with the beam and force a portion of the beam radially inwardly, which in turn brings the beam into impingement with a lead disposed between the beams, thereby longitudinally fixing the lead relative to the device. The components may be configured such that the beams impinge on the lead with sufficient force to also rotationally fix the lead relative to the device. Fixing the lead relative to the device may comprise impingement with force sufficient to inhibit translation or rotation relative to the device, although allowing translation or rotation upon application of large forces, or fixing may comprise impingement with force sufficient to prevent non-destructive translation or rotation relative to the device. The first prong wrench 43 is hollow, with a lumen 50 communicating through the handle portion 47, to allow the tool to be used to rotate the anchoring system while the lead is disposed in the lumen of the anchoring system, in a method in which the device is installed, assembled with the locking portion in the anchoring portion and the lead passing through both, with the entire device installed through a stereotactic frame. The second prong wrench 48 shown in FIG. 14 is an offset wrench which includes hollow partial cylinders tipped with prongs, with slots 49 along the length of the cylinders. This tool can be used to rotate either of the two portions when the lead is already disposed in the device. The offset configuration allows rotation of the components while a stereotactic frame that has been used to install the lead remains in place, so that the locking portion can be rotated without removing the frame used to install the device.

In use, a surgeon will cut through the scalp, set aside a flap of the scalp, and drill through the skull to create a burr hole of a diameter corresponding to the outer diameter of the anchoring portion. The device can be provided with the locking mechanism installed in the anchoring portion, and a surgeon can install the assembled device using a prong wrench operating on the notches of the anchoring portion, screwing the assembled device into the skull until it is flush with the superior surface of the skull. The surgeon will then pass the lead through the device and into the brain (preferably using a cannula over the lead). With the lead properly installed in the brain, the surgeon will rotate the locking portion using a prong wrench. The open sided prong wrench 48 may be used for this step because the lead is already in place, it may be passed through the side opening 49 while the prongs 46 are inserted into the locking portion wrench notches 37.

The installation can also be accomplished by first installing the anchoring portion and thereafter installing the locking portion. The surgeon can install the anchoring portion in the burr hole, with the proximal rim of the anchoring portion flush with the outer surface of the skull, using the prong wrench to screw the anchoring portion into the burr hole (the burr hole be threaded prior to this step). With the anchoring portion installed, the surgeon will install the locking portion into the anchoring portion, preferably with the lobes positioned, circumferentially, between the prongs, so that they do not impinge on the prongs or force them inwardly during insertion (this alignment is necessary if the lead is already in place, but not if the lead is not yet placed). The surgeon will insert the locking portion until the proximal surface of the locking portion is flush with the proximal end of the anchoring portion and the outer surface of the skull. (If the surgeon has already inserted the lead into the brain and through the anchoring portion, the surgeon may thread the lead through the locking portion and thereafter slip the locking portion over the lead and into the anchoring portion.) With the device assembled and installed, the surgeon will insert the lead into the brain, passing it through the device and into the brain until it is located as desired by the surgeon. With the lead properly installed, the surgeon will rotate the locking portion to bring the lobes into impingement with the beams, and force the beams into contact with the lead. The surgeon can do this by inserting the narrow prongs of the prong wrench into the notches of the locking portion. When the lead is no longer needed, the surgeon will counter-rotate the locking portion to move the lobes out of contact with the beams and release the beams from impingement with the lead. With the lead loosened, the surgeon will remove the lead from the brain, and remove the locking portion and anchoring portion from the skull. The burr hole can then be addressed as appropriate.

Thus, the method for securing an intracranial lead in the burr hole with the device described above entails (1) fixing the anchoring portion within the burr hole; (2) inserting a locking portion into the lumen of the anchoring portion (these steps may be accomplished in the manufacture of the device, rather when used by a surgeon); (3) inserting an intracranial lead into a lumen of the locking portion and anchoring portion (before or after insertion of the locking portion); (4) advancing an intracranial lead through the lumen of the locking portion to a target brain location; and (5) rotating the locking portion within the anchoring portion to move internal cam surfaces of the locking portion into impingement with at least one prong, thereby deflecting the prongs radially inward to engage the intracranial lead. If the device is not pre-assembled with the locking portion in the anchoring portion, the lead may be inserted prior to insertion of the locking portion.

While the preferred embodiments of the devices and methods have been described in reference to the environment in which they were developed, they are merely illustrative of the principles of the inventions. The elements of the various embodiments may be incorporated into each of the other species to obtain the benefits of those elements in combination with such other species, and the various beneficial features may be employed in embodiments alone or in combination with each other. Other embodiments and configurations may be devised without departing from the spirit of the inventions and the scope of the appended claims.

We claim:

1. An intracranial lead fixation device, for use with an intracranial lead configured for insertion into the brain of a patient, through a burr hole in the skull of the patient, comprising:
an anchoring portion 8 having an outer contour configured for placement in the burr hole, a distal end 10d and a proximal end 10p, and a circular lumen 14 extending from the distal end 10d to the proximal end 10p and a luminal wall 15 about the lumen 14, and a pair of prongs 16, 17 each having a prong distal end 16d, 17d and a prong proximal end 16p, 17p, wherein each prong distal end 16d, 17d is secured to the luminal wall proximate the distal end 10d of the anchoring portion 8 and each of the prongs 16, 17 comprise a beam 20, 21 which extends proximally within the lumen 14 of the anchoring portion 8 and terminating at the proximal end 10p of the anchoring portion 8;
a locking portion 9 having a circular outer contour configured for placement in the lumen of the anchoring portion, a distal end 31d and a proximal end 31p, and a locking portion lumen 31L extending from the distal end 31d to the proximal end 31p and a luminal wall 39 about the lumen 31L, wherein the lumen 31L has a first circumferential region having a major transverse diameter D1 and a second circumferential region having a minor transverse diameter D2 smaller than the major transverse diameter D1; wherein
the locking portion is disposed within the lumen of the anchoring portion, and the pair of prongs 16, 17 of the anchoring portion 8 are disposed within the lumen 31L of the locking portion 9,
whereby the locking portion may be rotated within the anchoring portion to move the second circumferential region into contact with each beam 20, 21 and force each beam to bend radially inwardly to trap a lead passing through the intracranial lead fixation device, and the locking portion may be counter-rotated to move the first circumferential region into apposition with the beams thereby allowing the beams to open and allow unimpeded passage of the lead through the intracranial lead fixation device.

2. The intracranial lead fixation device of claim 1, wherein:
each beam 20, 21 is resiliently deformable, such that after bending radially inwardly from a first radial position to trap the lead, the beams resiliently return to the first radial position upon rotation of the locking portion to move the second circumferential region out of contact with each beam 20, 21.

3. The intracranial lead fixation device of claim 1, wherein:
a second circumferential region having a minor transverse diameter D2 is established by internal lobes 40, 41 on the lumen luminal wall 39.

4. The intracranial lead fixation device of claim 1, wherein:
the lumen 31L of the locking portion has an elliptical transverse cross section.

5. The intracranial lead fixation device of claim 1, wherein:
the prongs 16, 17 each further comprise a radially outwardly projecting catch 22, 23 at a proximal end 20p, 21p of each beam 20, 21, and
the locking portion has an inner wall which comprises pockets 35, 36 configured to receive the catches.

6. The intracranial lead fixation device of claim 1, wherein:
the anchoring portion proximal end has a proximal rim 12, and the locking portion has a proximal rim 32, and each prong proximal end 16p, 17p terminates proximally at the same level as the proximal rim 12 of the anchoring portion, whereby the intracranial lead fixation device may be installed flush with a superior surface of the patient's skull.

7. The intracranial lead fixation device of claim 1, wherein:
each prong 16, 17 has a concave inner surface, configured to accommodate a round lead.

8. The intracranial lead fixation device of claim 1, further comprising:
external screw threads on the anchoring portion; and
a pair of prong wrench notches in the proximal end 10p of the anchoring portion.

9. The intracranial lead fixation device of claim 1, further comprising:
a second pair of prong wrench notches 37 in the proximal end 31p of the locking portion.

10. The intracranial lead fixation device of claim 1, further comprising:
an externally extending flange 27 disposed at the proximal end of the anchoring portion.

11. The intracranial lead fixation device of claim 1, further comprising:
releasable snap-fittings disposed at the distal end of the anchoring portion.

12. A method for securing an intracranial lead in a burr hole of a patient's skull, the method comprising:
providing the device of claim 1; and
drilling through the skull to create a burr hole of a diameter corresponding to the outer diameter of the anchoring portion;
with the locking portion installed in the anchoring portion, installing the assembled device into the burr hole
passing the lead through the device and into the brain; and
rotating the locking portion relative to the anchoring portion to move the minor transverse diameter D2 into contact with the prongs to thereby force the prongs into contact with the intracranial lead.

13. A method for securing an intracranial lead in a burr hole of a patient's skull, the method comprising:
providing an anchoring portion having an external fixation surface and an lumen containing at least one prong extending through the lumen;
fixing the anchoring portion within the burr hole;
inserting a locking portion into the lumen of the anchoring portion;
inserting an intracranial lead into a lumen of the locking portion;
advancing an intracranial lead through the lumen of the locking portion to a target brain location; and
rotating the locking portion within the anchoring portion to move internal cam surfaces of the locking portion into impingement with the at least one prong, thereby deflecting the prongs radially inward to engage the intracranial lead.
